(19) United States
(12) Patent Application Publication
(10) Pub. No.: US 2012/0213702 A1
(43) Pub. Date: Aug. 23, 2012

(54) NOVEL DIAGNOSTIC AGENTS FOR THE NON-INVASIVE IN VIVO IMAGING OF AMINOPEPTIDASE N (APN/CD13)

(76) Inventors: Christoph Bremer, Muenster (DE); Carsten Höltke, Steinfurt (DE)

(21) Appl. No.: 13/393,440

(22) PCT Filed: Aug. 31, 2010
(86) PCT No.: PCT/EP10/62669

§ 371 Date: May 11, 2012

(30) Foreign Application Priority Data
Sep. 1, 2009 (EP) 09169131.1

(51) Int. Cl. A61K 51/04; C07D 207/16; C07F 15/02; C07F 3/00; C07F 13/00; A61K 49/00; A61K 49/10; A61K 31/40; A61P 35/00; A61P 29/00; A61P 19/02; A61P 35/02; A61P 13/12; A61P 9/00; G01N 33/53; C07D 403/14

(52) U.S. Cl. 424/1.89; 548/427; 548/455; 548/533; 548/402; 534/14; 534/10; 424/9.6; 424/1.65; 424/9.361; 514/423; 435/7.8; 424/1.85

(57) ABSTRACT

The present invention relates to aminopeptidase N (APN) inhibitor conjugates of formula I wherein W is a —CO— or an —SO₂— group and at least one of R₁ or R₂ represents (OCH₂—CH₂)n-X, where n is an integer of 1 to 100 and X represents H or a detectable label or a therapeutic, and the other represents an alkoxy group or OH, and wherein R₃, R_{3′} and R_{3″} is independently selected from an alkoxy group or (OCH₂—CH₂)n-X, where n is an integer of 1 to 100 and X represents H or a detectable label or a therapeutic, and R₄ is selected from the group comprising: wherein R is an alkyl group. Furthermore, the present invention relates to a diagnostic and/or pharmaceutical composition comprising the conjugate of the invention. The present invention also relates to the conjugate or composition of the invention for use in the diagnosis or treatment of APN-associated diseases like for example angiogenesis in cancer, rheumatoid arthritis, leukemia and diabetic nephropathy, as well as to the use of the conjugate or of the composition of the invention in the diagnosis or treatment of angiogenesis in cancer, rheumatoid arthritis, leukemia and diabetic nephropathy. In a further aspect, the present invention relates to kits comprising the conjugate or composition of the invention. The conjugate or pharmaceutical/diagnostic composition of the invention can also be used in in vivo or in vitro imaging studies of aminopeptidase N.

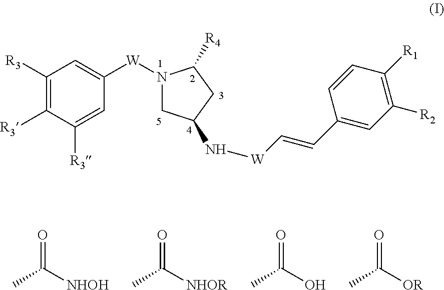

The present invention relates to aminopeptidase N (APN) inhibitor conjugates of formula I

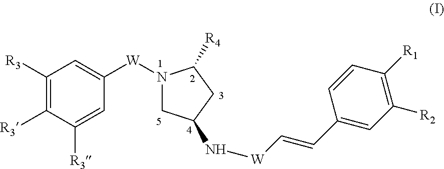

wherein W is a —CO— or an —SO₂— group and at least one of R₁ or R₂ represents (OCH₂—CH₂)n-X, where n is an integer of 1 to 100 and X represents H or a detectable label or a therapeutic, and the other represents an alkoxy group or OH, and wherein R₃, R₃′ and R₃″ is independently selected from an alkoxy group or (OCH₂—CH₂)n-X, where n is an integer of 1 to 100 and X represents H or a detectable label or a therapeutic, and R₄ is selected from the group comprising:

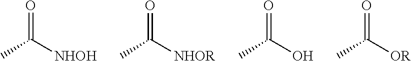

wherein R is an alkyl group.

Furthermore, the present invention relates to a diagnostic and/or pharmaceutical composition comprising the conjugate of the invention. The present invention also relates to the conjugate or composition of the invention for use in the diagnosis or treatment of “APN-associated diseases” like for example angiogenesis in cancer, rheumatoid arthritis, leukemia and diabetic nephropathy, as well as to the use of the conjugate or of the composition of the invention in the diagnosis or treatment of angiogenesis in cancer, rheumatoid arthritis, leukemia and diabetic nephropathy. In a further aspect, the present invention relates to kits comprising the conjugate or composition of the invention. The conjugate or pharmaceutical/diagnostic composition of the invention can also be used in in vivo or in vitro imaging studies of aminopeptidase N.

A variety of documents is cited throughout this specification. The disclosure content of said documents (including any manufacture's specifications, instructions, etc.) is herewith incorporated by reference; however, there is no admission that any document cited is indeed prior art as to the present invention.

### BACKGROUND OF THE INVENTION

Aminopeptidase N (APN/CD13) is a zinc-containing ectopeptidase involved in the degradation of neutral or basic amino acids (N-terminally). APN is known to be upregulated in affected tissues in a variety of diseases such as cancer, leukemia, diabetic nephropathy, and rheumatoid arthritis. Especially in the case of cancer, APN is related with a high degree of neovascularisation of the tumor. APN has been shown to be involved in cancer angiogenesis, invasion and metastasis. A dysregulation of APN expression occurs in diverse inflammatory diseases and in cancers.

Chen et al. (J. Labelled Cpd. Radiopharm. 43, 103-111, 2000) dislose the synthesis of 2(S)-benzyl-3-[hydroxyl(1′(R)-aminoethyl)phosphinyl]propanoyl-L-3-[¹²⁵I]-iodotyrosine, which are proposed to be suitable for the characterization of the biochemical and pharmacological properties of aminopeptidase N and its in vivo inhibition.

Further APN inhibitors are flavone-8-acetic acid derivatives the synthesis and characterization of which are disclosed in Bauvois et al., (J. Med. Chem. 46, 3900-3913, 2003).

Chen et al (J. Med. Chem. 43, 1398-1408, 2000) disclose dual phosphinic inhibitors representing a single molecule capable to block the active site of both neutral endopeptidase (NEP) and APN with nanomolar affinities.

Fournier-Zaluski et al (J. Med. Chem. 35, 1259-1266, 1992) disclose derivatives of amino acids bearing various zinc-coordinating moieties.

EP 1 346 729 describes peptide-based conjugates for treating or diagnosing a cardiovascular disease, wherein the CD13/APN homing molecule is based on specific peptides.

Bauvois et al (Med. Res. Rew. 26, No. 1, 88-130, 2006), as well as Xu and Li (Curr. Med. Chem.—Anticancer Agents, 5, 1-21, 2005) published review articles dealing with different classes of APN inhibitors. In particular, in the latter, a class of APN inhibitors based on derivatives of trans-4-aminoproline and their properties are described.

As mentioned above, the literature gives quite a number of examples of ligands affine to APN, but only very few structures are known which do not contain peptidic subunits. Since proteins and to an even greater extent small peptides are quickly metabolized in vivo there is a growing interest focused on small molecular non-peptidic substances (ligands) with a high affinity to the target structure. Also, only few ligands exhibit target affinities in the low or subnanomolar range, which is essential for imaging purposes.

Inhibitors based on trans-4-aminoproline substituted on one side with a benzyl derivative and on the other side with a cinnamoyl derivative are known. It was shown that the presence of hydroxy groups on the aromatic ring of the cinnamoyl moiety significantly increases the inhibitor potency of the compounds with respect to the equivalent compound bearing methoxy groups in the same positions, which clearly confirms the importance of hydrogen bond between inhibitors and the active site of the enzyme [Xu and Li (Curr. Med. Chem.—Anticancer Agents, 5, 1-21, 2005)].

The technical problem underlying the present invention was to provide non-peptidic ligands affine to APN and exhibiting target affinities in the subnanomolar range, which is essential for imaging purposes.

The solution to said technical problem is achieved by providing the embodiments characterized in the claims.

It must be noted that as used herein, the singular forms “a”, “an”, and “the” include plural references unless the context clearly indicates otherwise. Thus, for example, reference to “a reagent” includes one or more of such different reagents, and reference to “the method” includes reference to equivalent steps and methods known to those of ordinary skilled in the art that could be modified or substituted for the method described herein.

We have surprisingly found that derivatives of trans-4-aminoproline, wherein the cinnamoyl substituent bears an alkoxy group and/or a labeling compound, exhibit target affinities in the subnanomolar range. Accordingly, they are useful for imaging purposes. This is surprising because the presence of hydroxy groups on the aromatic ring of the cinnamoyl moiety significantly increases the inhibitor potency of the compounds which clearly confirms the importance of hydrogen bond between inhibitors and the active site of the enzyme [Xu and Li (Curr. Med. Chem.—Anticancer Agents, 5, 1-21, 2005)].

Thus, in a first aspect, the present invention relates to an aminopeptidase N inhibitor conjugate of formula I

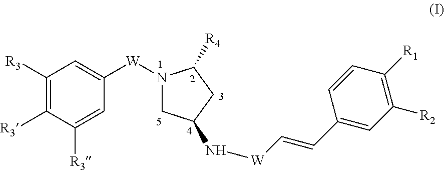

wherein W is a —CO— or an —SO₂— group and at least one of R₁ or R₂ represents (OCH₂—CH₂)n-X, where n is an integer of 1 to 100 and X represents H or a detectable label or a therapeutic, and the other represents an alkoxy group or OH, and wherein R₃, R₃′ and R₃″ is independently selected from an alkoxy group or (OCH₂—CH₂)n-X, where n is an integer of 1 to 100 and X represents H or a detectable label or a therapeutic, and R₄ is selected from the group comprising:

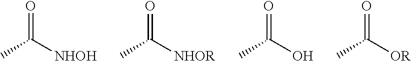

wherein R is an alkyl group.

In a preferred embodiment at least one of R₁ or R₂ represents (OCH₂CH₂)ₙ—X, wherein n is an integer of 1 to 100, and wherein X represents a detectable label or a therapeutic substance, the detectable label being preferred.

In another preferred embodiment R₃, R₃′ and R₃″represent three identical alkoxy groups (methoxy being preferred) and at least one of R₁ or R₂ represents (OCH₂CH₂)ₙ—X, wherein n is an integer of 1 to 100 (1 to 4 being preferred), X represents a detectable label or a therapeutic substance (the detectable label being preferred) and R₄ is defined as above.

In a more preferred embodiment of the invention W is —CO—.

In another preferred embodiment, the detectable label may be a fluorescent dye or a tautomer(s) thereof.

Furthermore, the present invention relates to a diagnostic and/or pharmaceutical composition comprising the conjugate of the invention. The present invention also relates to the conjugate or composition of the invention for use in the diagnosis or treatment of “APN-associated diseases” like for example angiogenesis in cancer, rheumatoid arthritis, leukemia and diabetic nephropathy, as well as to the use of the conjugate or of the composition of the invention in the diagnosis or treatment of angiogenesis in cancer, rheumatoid arthritis, leukemia and diabetic nephropathy. In a further aspect, the present invention relates to diagnostic or therapeutic kits comprising the first and second entity of the conjugates of the invention and/or the conjugate and/or composition of the invention and optionally instructions for use. The conjugate or pharmaceutical/diagnostic composition of the invention can also be used in in vivo or in vitro imaging studies of aminopeptidase N.

APN is known to be upregulated in affected tissues in a variety of diseases such as cancer, leukemia, diabetic nephropathy, and rheumatoid arthritis. It follows that “APN-associated disease” includes in its broadest sense all kinds of diseases which are characterized by an upregulation of APN, for example an upregulation on the protein level in the respective cells/tissues of a subject. “Upregulation” means, that the level of APN which is found/detected on mRNA and/or protein level is above the average when compared to non-affected tissue (for example tissue of a healthy patient or tissue from a non-affected part of the same patient). Especially in the case of cancer, APN is related with a high degree of neovscularisation of the tumor. APN has been shown to be involved in cancer angiogenesis, invasion and metastasis. A dysregulation of APN expression occurs in diverse inflammatory diseases and in cancers, where a inhibitor of APN furnished with a fluorescent, MR-active or radioactive label may serve as a tool for diagnosis. All these diseases/conditions are included by the term “APN-associated diseases”. APN is likewise highly expressed in at least the following tissues (a) cardiovascular related tissues: heart atrium (right), heart ventricle (left), artery, HUVEC cells, fetal liver, liver, liver liver cirrhosis, thrombocytes, fetal kidney, kidney; (b) liver tissues: fetal liver, liver, liver cirrhosis; (c) kidney tissues, fetal kidney, kidney (expression in kidney tissues demonstrates that the activity of the ANP can be modulated to diagnose/treat for example blood pressure disorders as hypertension or hypotension); (d) the hematological system: leukocytes (peripheral blood), thrombocytes. (the activity of the ANP can therefore be modulated to diagnose/treat hematological disorders); (e) tissues of the gastroenterological system: small intestine, ileum, fetal liver, liver, liver liver cirrhosis, HEP G2 cells (the expression in the above mentioned tissues and in particular the differential expression between diseased tissue liver liver cirrhosis and healthy tissue liver demonstrates that the ANP can be utilized to diagnose and/or treat gastroenterological disorders; (f) the endocrinological system: pancreas, pancreas liver cirrhosis (the expression in the above mentioned tissues and in particular the differential expression between diseased tissue pancreas liver cirrhosis and healthy tissue pancreas demonstrates that the ANP can be utilized to diagnose/treat endocrinological disorders. It follows that diseases that are characterized by an upregulation of APN in the above mentioned cells and/or tissues are “ANP-associated diseases”.

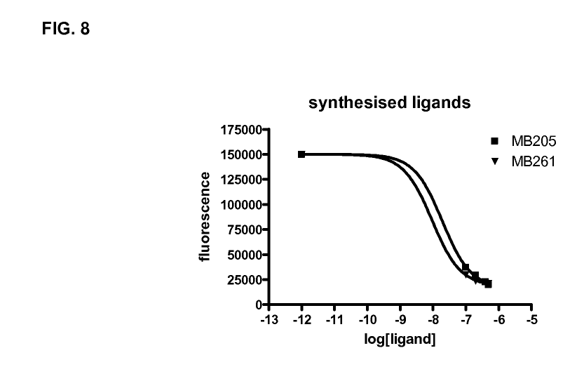

An “APN inhibitor conjugate” or “conjugate” is herein defined as a molecule as depicted in Formula (I) and defined herein, that specifically binds to aminopeptidase N (APN). As used herein, the terms “specific binding” or “high target affinity” are exchangeable and means binding that is measurably different from a non-specific interaction. Specific binding can be measured, for example, by determining binding of a molecule compared to binding of a control molecule, which is generally a molecule of similar structure that does not have binding activity, for example, a compound of similar size that lacks a specific binding sequence. Specific binding is present if the molecule has measurably higher affinity for the ligand affine to APN than the control molecule. Specificity of binding can be determined, for example, by competition with a control molecule that is known to bind to a target, e.g., bestatin. Preliminary inhibition assays with the precursor compounds MB205 and MB261 have been carried out (FIG. 8), resulting in IC₅₀ values of 19.4 nM and 9.4 nM, respectively. In a similar experiment the affinity of bestatin has been evaluated to be 2.3 μM. The modification of the conjugates of the invention towards the mentioned hydroxamic acid derivatives should yield a further enhancement of affinity.

It is thus envisaged that the APN inhibitor conjugates of the present invention (i.e. the compounds of the present invention which are sometimes also denoted as high affinity ligands or ligands affine to APN) specifically bind to aminopeptidase N (APN), i.e. they are characterized by an IC₅₀ in the subnanomolar range, i.e. in the range of 20 nM or lower, for example 19, 18, 17, 16, 15, 14, 13, 12, 11, 10, 9, 8, 7, 6, 5, 4, 3, 2, 1, 0, 1 nM or even lower IC50 values, for example in an experimental set up as explained in the appended Example 2 and 3.

The term “specific binding” or “target affinity” as used herein, includes high affinity specific binding. Specific binding can be exhibited, e.g. by a high affinity ligand affine to APN, for example, a ligand affine to APN having a K_{d} of at least about of 10⁻⁷ M, at least about 10⁻⁸ M, at least about 10⁻⁹ M, at least about 10⁻¹⁹ M, or can have a K_{d} of at least about 10⁻¹¹ M or 10⁻¹² M or greater. The high affinity ligands of the present invention are useful for targeting angiogenic vasculature.

Large molecules/conjugates of the invention (i.e., wherein n=25-100) will exhibit a “blood pool” effect which is considered to be advantageous for specific body compartments or tumor pathology, respectively. Small molecules/conjugates of the invention (i.e., where n is an integer of about 1 to 10, preferably 1 to 4) will be excreted more rapidly from the organism which is advantageous for in vivo use.

The term “alkoxy” as used herein is meant to include linear or branched C₁-C₁₂, preferably C₁-C₆ alkoxy; e.g., methoxy, ethoxy, iso-propoxy, n-propoxy, iso-butoxy, n-butoxy, sec.-butoxy, tert.-butoxy, n-pentoxy, iso-pentoxy, sec.-pentoxy, tert.-pentoxy, neo-pentoxy, n-hexoxy, 1-methylpentoxy, 2-methylpentoxy, 3-methylpentoxy, 4-methylpentoxy, 1-ethylbutoxy, 2-ethylbutoxy, 1,1-dimethylbutoxy, 2,2-dimethylbutoxy or 3,3-dimethylbutoxy. Methoxy is preferred.

The term “alkyl” as used herein is meant to include linear or branched C₁-C₁₂, preferably C₁-C₆. Preferably the alkyl is —CH₃.

A particularly advantageous method of conjugation may be applied when the polyethylene glycol (PEG) derivative is introduced during the synthesis of the intermediate. In more detail, the PEG derivative is coupled to the commercially available starting compounds caffeic acid or the monomethoxy derivatives thereof, and bears on the other terminus a reactive group necessary for binding of “X”, wherein “X” is for example the detectable label. In a final stage of the process the obtained intermediate is then reacted with an aminoproline derivative, which is substituted with a benzyl derivative and a zinc-coordinating moiety.

In a preferred embodiment the conjugates of the invention have the following substituents: R₁ is OCH₃ and R₂ is (OCH₂—CH₂)n-X and R₃ is 3,4,5-(OCH₃)₃.

In a further preferred embodiment of the invention R₃ is 3,5-(OCH₃)₂-4-(OCH₂—CH₂)n-X, wherein “X” is a detectable label or a therapeutic (a detectable label being preferred), and R₁ and R₂ are OCH₃.

The present invention particularly prefers compounds of the Formula I, wherein n is 1 or 2, 3, 4, 5, 6, 7, 8, 9, 10 or n is 1 to 4 or 1 to 5, 1 to 10, 1 to 15, 1 to 20, 1 to 25, 1 to 30, 1 to 40, 1 to 50, 1 to 60, 1 to 70, 1 to 80, 1 to 90, 1 to 100. More preferably, n is 1 to 4.

It is envisaged that the “detectable label” of the present invention includes a radioactive, colored, bioluminescent, chemiluminescent, fluorescent or phosphorescent label.

In a preferred embodiment, the detectable label is a fluorescent dye or a tautomer(s) thereof. The absorption maximum of the fluorescent dye is preferably from about 600 nm to 900 nm.

In a preferred embodiment of the compound of the invention the fluorescent dye is selected from Cy 5, Cy 5.5, Cy 7, C 3, Cy 3.5, flourescein (FITC), heptamethylene thiocyanine, ROX, TAMRA, CAL Red, Red 640, FAM, TET, HEX, Oregon Green, TRITC, APC, DY-751, ATTO 740, ATTO 725 and ATTO 700.

The fluorescent dyes as described herein are well-known to the skilled person and furthermore commercially available, e.g., at Amersham Bioscience Europe GmbH, Freiburg, Germany; Dyomics GmbH, Jena, Germany; MoBiTe GmbH, Gottingen, Germany; Invitrogen GmbH, Karlsruhe, Germany.

A “therapeutic” as used herein (i.e. a compound of the Formula I wherein at least one X is a therapeutic) includes any kind of therapeutically active agent which might exert a beneficial effect on the APN-associated diseases as defined herein. It is for example envisaged to couple the compounds of Formula I to an agent/drug (X) which reduces the proliferation of tumor cells; triggers immune cells etc. In this regard, it will be understood that the wanted therapeutic effect of the therapeutic of the invention (i.e. when X is a therapeutic) is dependent on the APN-associated disease to be treated.

It is preferred that the conjugates of the present invention comprise at least one “X” which is a detectable label. In a more preferred embodiment R1 and/or R2 are X, wherein X is represented by a detectable label.

In a more preferred embodiment the conjugate of the invention is selected from

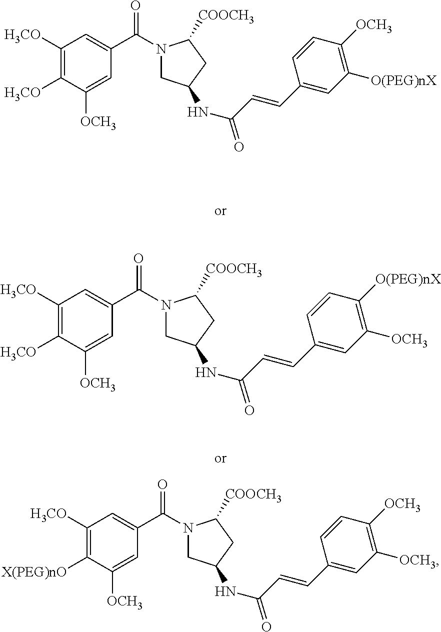

“n” is defined herein elsewhere—in the context of this specific embodiment, “n” is preferably 4 and “X” is preferably a near-infrared fluorescent dye like Cy 5.5.

The present invention also relates to the use of the first entity of the conjugates of the invention (Formula I), and the second entity “X” for the manufacture of the conjugates of the invention. The present invention also relates to the use of the conjugates of the present invention for the preparation/manufacture of a diagnostic and/or pharmaceutical composition. The conjugates and/or entities (first entity and second entity) of the present invention may also be used for the preparation/manufacture of a diagnostic or therapeutic kit.

### Imaging Methods

The compounds of the invention are preferably employed for fluorescence mediated tomography (FMT). This imaging technique allows a three-dimensional, quantitative reconstruction of fluorochrome distribution in vivo. FMT can, e.g., be applied to detect and quantify fluorophores accumulated in deep seated breast tissue.

Moreover the targeted fluorochrome can be applied for different fluorescence reflectance imaging (FRI) techniques, which provide a surface weighted image of tissue fluorescence. FRI is a rapid technique that does not require image reconstruction. It can be miniturazied and thus incorporated into endoscopse, catheters or applied intra-operatively.

The term “fluorescent mediated tomography” has a well-recognized meaning and is further explained herein.

Altering the labelling and detection strategy towards optical imaging techniques like fluorescence reflectance imaging (FRI), fluorescence mediated tomography (FMT) or near infrared fluorescence (NIRF) imaging is an interesting alternative to the use of nuclear imaging techniques like single photon emission computed tomography (SPECT) or positron emission tomography (PET). While FRI is limited to superficial tissue structures, FMT offers the possibility of 3D quantitative imaging of photon absorption of tissue in vivo combining these optical imaging techniques with targeted fluorescent probes offers excellent signal to noise ratios (SNRs) and thus very high sensitivity to detect molecular structures (such as cell receptors). Optical imaging techniques allow to delineate structures in the picomolar (10⁻¹²) range, which is comparable to conventional nuclear imaging techniques and about 6 orders of magnitude more sensitive compared to MRI. Besides high SNRs imaging in the near infrared shows very efficient tissue penetration as the absorption by water and haemoglobin is relatively low (“diagnostic window”).

The term “detectable label” as used herein refers to compounds which allow the detection, preferably the detection of their location in vivo, with the techniques described herein.

Particularly preferred as detectable label are: (1) chelated paramagnetic metal ions such as Gd, Dy, Fe, and Mn, especially when chelated by macrocyclic chelant groups (e.g. tetraazacyclododecane chelants such as DOTA, D03A, HP-DO3A and analogues thereof) or by linker chelant groups such as DTPA, DTPA-BMA, EDTA, and DPDP; superparamagnetic iron oxide crystals; (1) vesicles containing fluorinated gases (i.e. containing materials in the gas phase at 37 DEG C which are fluorine containing e.g. SF₆ or perfluorinated C1-6 hydrocarbons or other gases and gas precursors); (2) chelated heavy metal cluster ions (e.g. W or Mo polyoxoanions or the sulphur or mixed oxygen/sulphur analogs); (3) covalently bonded non-metal atoms which are either high atomic number (e.g. iodine); and (4) iodinated compound containing vesicles.

Preferred paramagnetic metal ions include ions of transition and lanthanide metals (e.g. metals having atomic numbers of 6 to 9, 21-29, 42, 43, 44, or 57-71), in particular ions of Cr, V, Mn, Fe, Co, Ni, Cu, La, Ce, Pr, Nd, Pm, Sm, Eu, Gd, Tb, Dy, Ho, Er, Tm, Yb and Lu, especially of Mn, Cr, Fe, Gd and Dy, more especially Gd. Preferred heavy metal-containing detactable label may include atoms of Mo, Bi, Si, and W, and in particular may be polyatomic cluster ions (e.g. Bi compounds and W and Mo oxides). The metal ions are desirably chelated by chelant groups in the imaging moiety or in or on an imaging particle, (e.g. a vesicle or a porous or non-porous inorganic or organic solid), in particular linear, macrocyclic, terpyridine and N₂S₂ chelants, such as for example DTPA, DTPA-BMA, EDTA, D03A, TMT.

As it is well known, a “chelating agent” is a compound containing donor atoms that can combine by coordinate bonding with a metal atom to form a cyclic structure called a chelation complex or chelate.

In another preferred embodiment of the invention, the detectable label is Fe-oxide or Gd-chelate. These detectable labels are preferably used in MR-imaging.

Preferred non-metal detectable label include non zero nuclear spin atoms such as ¹⁸F, and heavy atoms such as ¹²⁵I. Such detectable label may be covalently bonded to a linker backbone, either directly using conventional chemical synthesis techniques or via a supporting group, e.g. a triiodophenyl group. As with the metal chelates discussed above, such non-metal atomic detectable label may be directly linked to a ligand affine to APN.

In a preferred embodiment of the invention, the detectable label is a ^{99m}Tc, ^{94m}Tc ⁶⁸Ga, ¹¹¹In, 62 ^{Cu} or ⁶⁴Cu chelator or from the group of radioactive compounds such as ¹⁸F, ¹³N, ⁷⁶Br, ¹¹C, ¹²³I, ¹²⁴I, or ¹²⁵I.

It is particularly preferred that the compounds of the invention and uses as described herein are employed for fluorescent mediated tomography or fluorescence reflectance imaging methods (i.e., surface weighted fluorescence reflectance imaging).

In a further preferred embodiment, the conjugates of the invention are used for scintigraphic in vivo imaging techniques like single photon emission computed tomography (SPECT) or positron emission tomography (PET)

In another preferred embodiment, the conjugates of the invention are used for in vitro imaging. Preferably, the in vitro imaging technique used is selected from immune histochemistry, Western-blotting, autoradiography.

### Compositions

In a further aspect, the present invention relates to a pharmaceutical and/or diagnostic composition comprising a conjugate of the invention as described herein and optionally a pharmaceutically and/or therapeutically acceptable carrier. In the context of the present invention “carrier” or “pharmaceutically acceptable carrier” or “diagnostic acceptable carrier” are used interchangeable.

Examples of carriers are well known in the art and include phosphate buffered saline solutions, water, emulsion, such as oil/water emulsions, various types of wetting agents, sterile solutions etc. Compositions comprising such carriers can be formulated by well known conventional methods. The compositions according to the invention can be administered to the subject at a suitable dose.

The dosage regime will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

The compositions of the invention may be used for diagnostic in vivo imaging methods and/or for therapeutic applications.

It is also envisaged that the conjugates of the present invention comprise one or two detectable label and additionally a therapeutic, or one or two therapeutic and additionally a detectable label. Conjugates comprising one detectable label and one therapeutic are likewise envisaged.

In the therapeutic and diagnostic compositions and/or uses and/or methods of the invention, the conjugates of the invention are administered in purified form together with a pharmaceutical carrier. The preferred form depends on the intended mode of administration and therapeutic or diagnostic application. The pharmaceutical carrier can be any compatible, nontoxic substance suitable to deliver the conjugates to the patient. Pharmaceutically acceptable carriers are well known in the art and include, for example, aqueous solutions such as (sterile) water or physiologically buffered saline or other solvents or vehicles such as glycols, glycerol, oils such as olive oil or injectable organic esters, alcohol, fats, waxes, and inert solids may be used as the carrier. A pharmaceutically acceptable carrier may further contain physiologically acceptable compounds that act, e.g. to stabilise or to increase the absorption of the conjugate. Such physiologically acceptable compounds include, for example, carbohydrates, such as glucose, sucrose or dextrans, antioxidants, such as ascorbic acid or glutathione, chelating agents, low molecular weight proteins or other stabilisers or excipients. One skilled in the art would know that the choice of a pharmaceutically acceptable carrier, including a physiologically acceptable compound, depends, for example, on the route of administration of the composition. Pharmaceutically acceptable adjuvants, buffering agents, dispersing agents, and the like, may also be incorporated into the pharmaceutical compositions.

For oral administration, the active ingredient can be administered in solid dosage forms, such as capsules, tablets, and powders, or in liquid dosage forms, such as elixirs, syrups, and suspensions. Active component(s) can be encapsulated in gelatin capsules together with inactive ingredients and powdered carriers, such as glucose, lactose, sucrose, mannitol, starch, cellulose or cellulose derivatives, magnesium stearate, stearic acid, sodium saccharin, talcum, magnesium carbonate and the like. Examples of additional inactive ingredients that may be added to provide desirable color, taste, stability, buffering capacity, dispersion or other known desirable features are red iron oxide, silica gel, sodium lauryl sulfate, titanium dioxide, edible white ink and the like. Similar diluents can be used to make compressed tablets. Both tablets and capsules can be manufactured as sustained release products to provide for continuous release of medication over a period of hours. Compressed tablets can be sugar coated or film coated to mask any unpleasant taste and protect the tablet from the atmosphere, or enteric-coated for selective disintegration in the gastrointestinal tract. Liquid dosage forms for oral administration can contain colouring and flavouring to increase patient acceptance.

The conjugates are however preferably administered parentally. Preparations of the conjugates for parental administration must be sterile. Sterilisation is readily accomplished by filtration through sterile filtration membranes, optionally prior to or following lyophilisation and reconstitution. The parental route for administration of conjugates is in accord with known methods, e.g. injection or infusion by intravenous, intraperitoneal, intramuscular, intraarterial or intralesional routes. The conjugates may be administered continuously by infusion or by bolus injection. A typical composition for intravenous infusion could be made up to contain 100 to 500 ml of sterile 0.9% NaCl or 5% glucose optionally supplemented with a 20% albumin solution and 10 μg to 250 mg of the conjugate, depending on the type of conjugate and its required dosing regime. Methods for preparing parenterally administrable compositions are well known in the art.

For the diagnostic or therapeutic methods disclosed herein, an effective amount of the conjugate of the invention must be administered to the subject. As used herein, the term “effective amount” means the amount of the conjugate that produces the desired effect. An effective amount often will depend on the moiety linked to the ligand affine to APN. An effective amount of a particular molecule/moiety for a specific purpose can be determined using methods well known to those in the art.

The present invention also relates to the use of a conjugate of the invention for the preparation of a diagnostic composition for the use in in vivo imaging of aminopeptidase N. Preferably the use in in vivo imaging is in mammals, more preferably in humans.

The present invention also relates to the use of a conjugate of the invention for the preparation of a composition the use in the diagnosis or therapy of cancer, the evaluation of cancer biology and/or monitoring of anti-cancer therapy.

The conjugates and/or diagnostic or therapeutic composition according to the invention may be used for in vivo imaging and/or simultaneous in vivo imaging and therapy.

In a preferred embodiment said diagnosis includes angiogenesis in cancer, rheumatoid arthritis, leukemia, inflammatory diseases and diabetic nephropathy.

In a preferred embodiment, said cancer is selected from breast cancer, ovarian cancer, cervical cancer, prostate cancer, melanoma, saracomas, lymphomas, bone malignancies, renal cancer, pancreas cancer, gastric cancer, thyroid cancer, lung cancer, colon cancer, Karposi's sarcoma and CNS tumors.

Another embodiment of the invention relates to a diagnostic method for diagnosing APN-associated diseases such as angiogenesis in cancer and inflammatory diseases, cancer, rheumatoid arthritis, leukemia or diabetic nephropathy comprising the step:
(a) administering an effective amount of the conjugate of the invention or the composition of the invention to a subject; and
(b) detecting upregulation of/upregulated aminopeptidase N.

In another embodiment, the invention relates to a therapeutic method for treating APN-associated diseases such as angiogenesis in cancer and inflammatory diseases, cancer, rheumatoid arthritis, leukemia or diabetic nephropathy comprising the step of administering an effective amount of the conjugate of the invention or the composition of the invention to a subject in need thereof, particularly to a patient suffering from or suspected to suffer from an APN-associated disease.

In a further embodiment the present invention relates to a kit comprising a conjugate, and/or composition, and/or the two entities forming the conjugates of the invention, and optionally a carrier, solvent, diluent, buffer for stabilizing and/or storing the inventions compounds. Said kit may further comprise instruction manuals which guide the skilled person in carrying out the detection methods which are inter alia described herein (e.g., diagnosis of cancer, the evaluation of cancer biology and/or monitoring of anti-cancer therapy).

Preferably, the instruction manual comprises information concerning the dosage and the administration of the compound and/or of the composition according to the invention.

The dosage regime utilizing the inhibitors or screened compounds (inhibitors) of the present invention is selected in accordance with a variety of factors including type, species, age, weight, sex and medical condition of the patient; the severity of the condition to be treated; the route of administration; and the particular compound employed. It will be acknowledged that an ordinary skilled physician or veterinarian can easily determine and prescribe the effective amount of the compound and required to prevent, counter or arrest the progress of the condition.

The instruction manual preferably comprises the information regarding the administration of the compound, more preferably, the instruction manual comprises details as set forth above in the various embodiments of the invention.

The figures show:

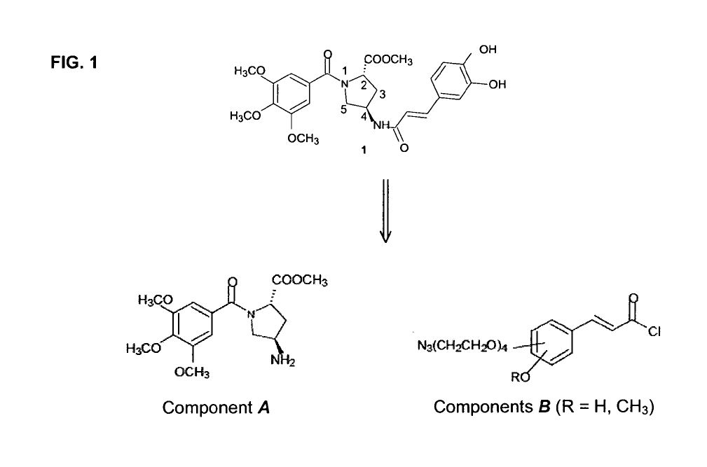

FIG. 1 Convergent synthesis of compound/with a proline derivative as component A and different spacer-modified caffeic acid derivatives as components B

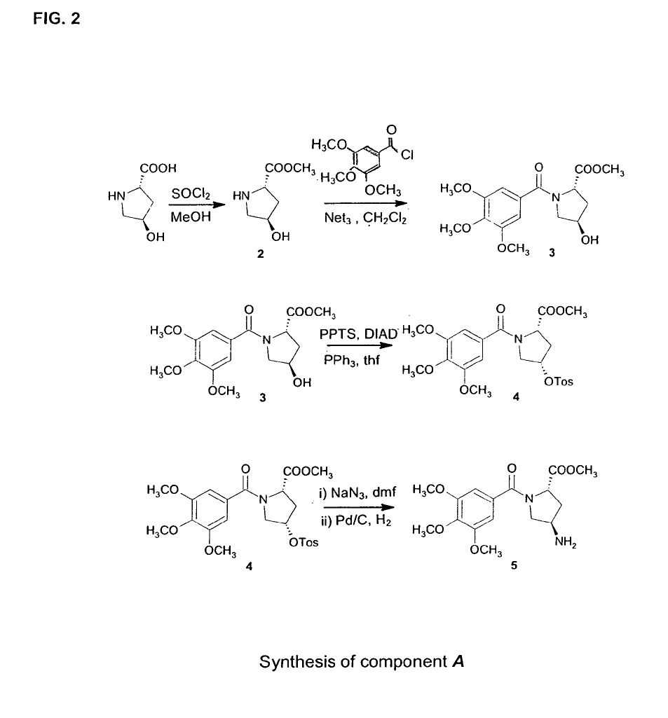

FIG. 2 Synthesis of component A

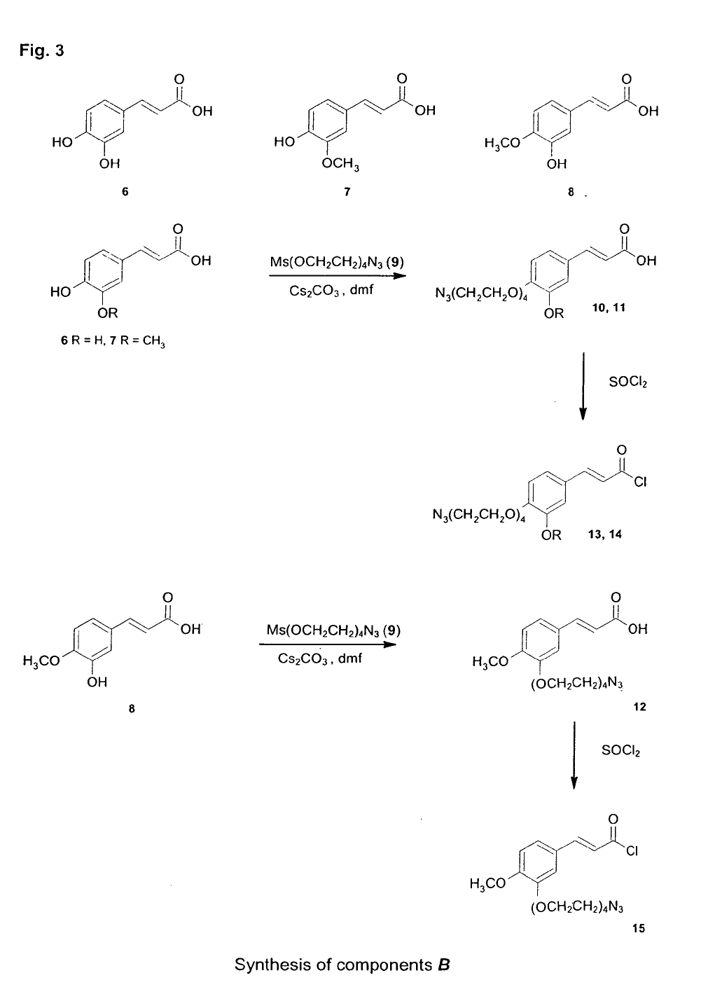

FIG. 3 Synthesis of component B

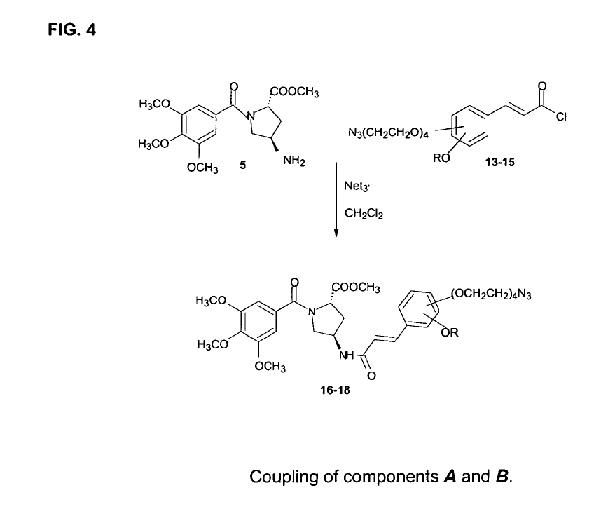

FIG. 4 Coupling of components A and B

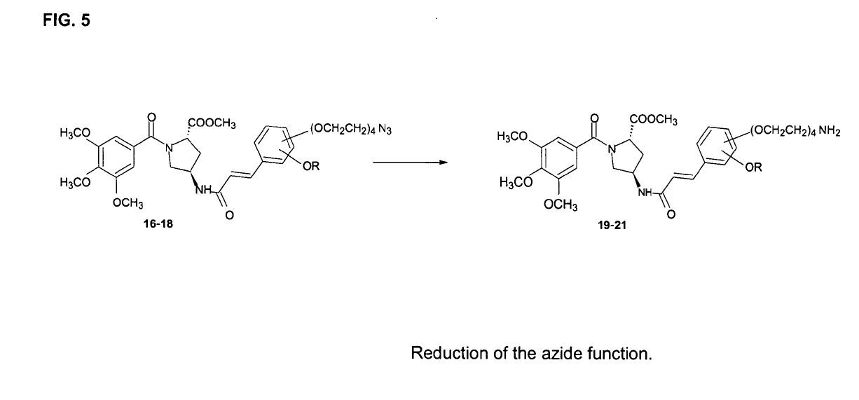

FIG. 5 Reduction of azide function

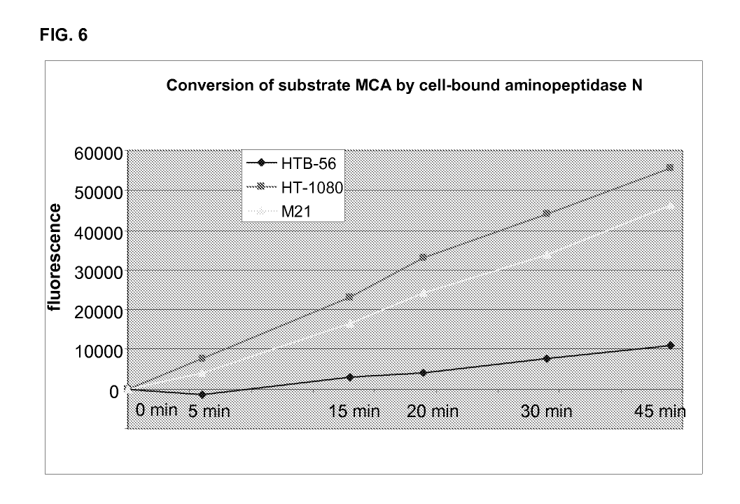

FIG. 6 Cell assay with cell binding APN on different cell lines

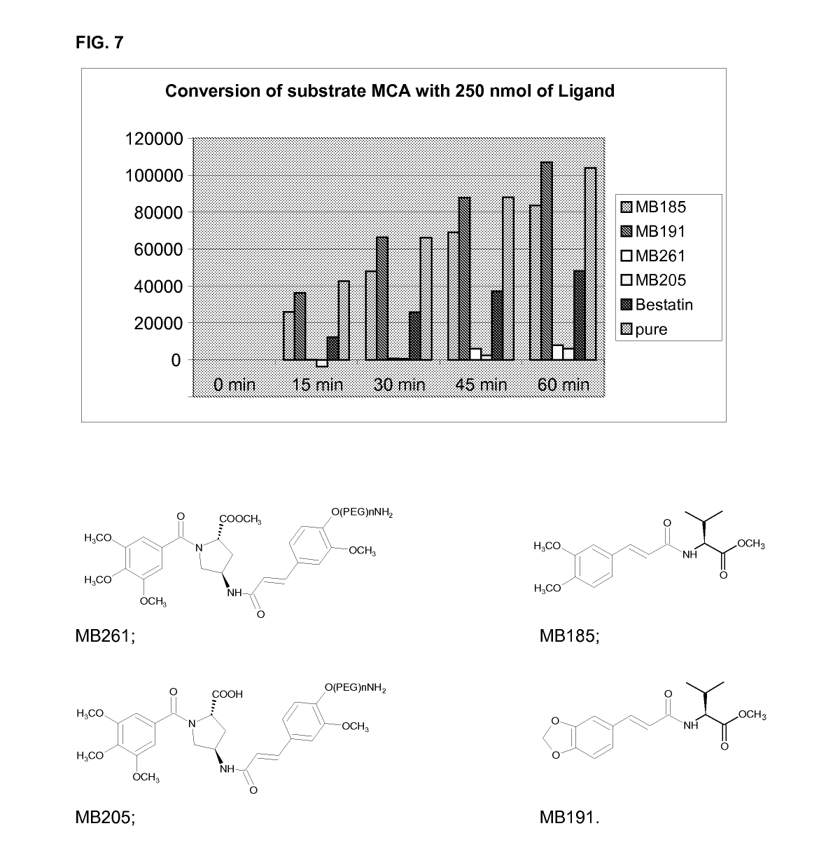

FIG. 7 Blocking effect of different substances affine to APN cell binding

FIG. 8 Inhibition binding assay with the synthesised precursor compounds MB205 and MB261, yielding IC₅₀ values of 19.4 nM and 9.4 nM, respectively.

### EXAMPLES

### Example 1

### Synthesis of a Variety of Fluorescently Labeled Derivatives of 1

Compound 1 is known to be a very powerful inhibitor of aminopeptidase N (APN, CD13, Xu W. F. 226^{th} ACS National Meeting 2003, 7). Herein a route for the synthesis of a variety of fluorescently labeled derivatives of 1 is described (see FIG. 1).

A convergent synthesis was chosen with a proline derivative as component A (see FIG. 2) and different spacer-modified caffeic acid derivatives as components B (see FIG. 3).

Starting with commercially available L-4-hydroxyproline (5.0 g, 38.1 mmol) the synthesis of component A was easily accomplished. The first step was esterification of the carboxylic acid function by reaction with thionyl chloride in methanol (150 ml, 10% (v/v)) to yield L-4-hydroxy proline methyl ester 2 (6.8 g, 98%). Reaction with 3,4,5-trimethoxybenzoylchloride (9.0 g, 39.0 mmol) leads to amide 3 (8.1 g, 61%), which is further converted to the C-4-inverted tosylate 4 by mitsunobu reaction with pyridinium-4-toluenesulfonate (PPTS), triphenylphosphine and diisopropyl azodicarboxylate (DIAD) in THF (only 24% yield). Alternatively, an Appel-Reaktion with triphenylphosphine and carbon tetrabromide yields the C-4-inverted bromide (yield≈60%). Again, inversion at C-4 was achieved. Substitution with sodium azide in DMF and subsequent reduction of the azide functional group by catalytic hydrogenation yields L-4-aminoproline 5 (component A) with restored stereochemistry on C-4 (two steps, 60% yield).

Components B can be synthesised starting from caffeic acid 6 and the monomethoxy derivatives 7 and 8 respectively, which are all commercially available. The route is outlined in FIG. 3. The PEG derivative 9 is already well established in our group and is available on multigram scale. Reaction of 9 with the caffeic acid derivatives 6-8 results in the PEGylated compounds 10-12 (≈90% yield). Subsequent chlorination with thionyl chloride gives the three desired components B (13-15).

The coupling of the components is achieved by reaction in dichloromethane with triethylamine as base, leading to the three different spacer-modified compounds 16-18 (FIG. 4). Alternatively, the coupling of the two components can be achieved by using common peptide coupling reagents like BOP, CU or EDC, with the carboxylic acids 10-12. The azide function of the coupling products can easily be reduced to the free amine by Staudinger reaction yielding amines 19-21, which can directly be used for coupling to fluorescent dyes like Cy5.5 or Cy7 (FIG. 5).

### Example 2

Surface aminopeptidase activity of intact human fibrosarcoma cells (HT-1080), human lung adenocarcinoma cells (HTB-56) and human melanoma cells (M-21) was measured by plating 15000 cells/well in a 96-well plate. Cells were allowed to recover for 24 h at 37° C. in MEM supplemented with 10% fetal calf serum, penicillin and streptomycin in a 5% CO₂ humidified air atmosphere. Following two washing steps with PBS the cells were incubated with 10 μM L-alanine-4-methyl-coumarinylamide-(MCA)-trifluoroacetate in PBS with 0.5% BSA, 20 mM HEPES (pH 7,2) and 2% DMSO.

The release of fluorescent product 7-amido-4-methylcoumarin was monitored on a Perkin Elmer Fusion plate reader with λ_{exc} of 330 nm and λₑₘ of 460 nm after different time points. Controls without enzyme/cells were used for background subtraction (FIG. 6).

To assay for inhibition of enzymatic activity due to binding of APN/CD13 specific ligands, cells were additionally incubated with the respective inhibitor with concentrations ranging from 0.5 nM to 1.0 mM. FIG. 7 shows the inhibition of fluorescence substrate generation using 250 nmol/well of the rescpective ligands (FIG. 7). Bestatin as a commercially available APN substrate with known affinity was used for comparison.

### Example 3

The IC₅₀ values of two different ligands (MB205 and MB261) were determined using nonlinear regression analysis of the above determined fluorescence values (GraphPad 4.0 Software) based on a one-site binding model. FIG. 8 shows binding curves of MB205 and MB261.

It will be clear that the invention may be practiced otherwise than as particularly described in the foregoing description and examples. Numerous modifications and variations of the present invention are possible in light of the above teachings and, therefore, are within the scope of the appended claims.

The entire disclosure of each document cited (including patents, patent applications, journal articles, abstracts, laboratory manuals, books, or other disclosures) in the Background of the Invention, Detailed Description, and Examples is incorporated herein by reference

### REFERENCES

Arap W, Pasqualini R, Ruoslahti E (1998) Cancer treatment by targeted drug delivery to tumor vasculature in a mouse model. Science 279: 377-380
Bauvois B, Dauzonne D (2006) Aminopeptidase-N/CD13 (EC 3.4.11.2) inhibitors: chemistry, biological evaluations, and therapeutic prospects. Med Res Rev 26: 88-130
Chang Y W, Chen S C, Cheng E C, Ko Y P, Lin Y C, Kao Y R, Tsay Y G, Yang P C, Wu C W, Roffler S R (2005) CD13 (aminopeptidase N) can associate with tumor-associated antigen L6 and enhance the motility of human lung cancer cells. Int Cancer 116: 243-252
Curnis F, Arrigoni G, Sacchi A, Fischetti L, Arap W, Pasqualini R, Corti A (2002) Differential binding of drugs containing the NGR motif to CD13 isoforms in tumor vessels, epithelia, and myeloid cells. Cancer Res 62: 867-874
Grifman M, Trepel M, Speece P, Gilbert L B, Arap W, Pasqualini R, Weitzman M D (2001) Incorporation of tumor-targeting peptides into recombinant adeno-associated virus capsids. Mol Ther 3: 964-975
Murakami H, Yokoyama A, Kondo K, Nakanishi S, Kohno N, Miyake M (2005) Circulating aminopeptidase N/CD13 is an independent prognostic factor in patients with non-small cell lung cancer. Clin Cancer Res 11: 8674-8679
Pasqualini R, Koivunen E, Kain R, Landenranta J, Sakamoto M, Stryhn A, Ashmun R A, Shapiro L H, Arap W, Ruoslahti E (2000) Aminopeptidase N is a receptor for tumor-homing peptides and a target for inhibiting angiogenesis. Cancer Res 60: 722-727
Rocken C, Licht J, Roessner A, Carl-McGrath S (2005) Canalicular immunostaining of aminopeptidase N(CD13) as a diagnostic marker for hepatocellular carcinoma. J Clin Pathol 58: 1069-1075
Yokoyama Y, Ramakrishnan S (2005) Addition of an aminopeptidase N-binding sequence to human endostatin improves inhibition of ovarian carcinoma growth. Cancer 104: 321-331
Zhang Z, Harada H, Tanabe K, Hatta H, Hiraoka M, Nishimoto S (2005) Aminopeptidase N/CD13 targeting fluorescent probes: synthesis and application to tumor cell imaging. Peptides 26: 2182-2187
Zhang Z, Hatta H, Tanabe K, Nishimoto S (2005) A new class of 5-fluoro-2′-deoxyuridine prodrugs conjugated with a tumor-homing cyclic peptide CNGRC by ester linkers: synthesis, reactivity, and tumor-cell-selective cytotoxicity. Pharm Res 22: 381-389

1. An aminopeptidase N inhibitor conjugate of formula I 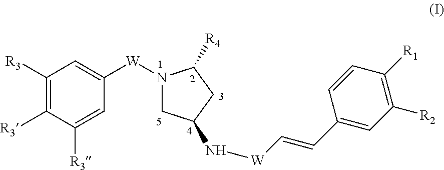
wherein W is a —CO— or an —SO₂— group and at least one of R₁ or R₂ represents (OCH₂—CH₂)ₙ—X, where n is an integer of 1 to 100 and X represents H or a detectable label or a therapeutic, and the other represents an alkoxy group or OH, and wherein R₃, R₃′ and R₃″ is independently selected from an alkoxy group or (OCH₂—CH₂)ₙ—X, where n is an integer of 1 to 100 and X represents H or a detectable label or a therapeutic, and R₄ is selected from the group comprising: 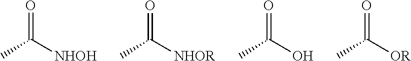
wherein R is an alkyl group.

2. The conjugate of claim 1 wherein R₃, R₃′ and R₃″ are 3,4,5-(OCH₃)₃ and at least one of R₁ or R₂ represents (OCH₂—CH₂)ₙ—X.

3. The conjugate of claim 1 wherein W is —CO—.

4. The conjugate of claim 1 wherein R₁ is OCH₃.

5. The conjugate of claim 1, wherein n is 1.

6. The conjugate of claim 1, wherein the detectable label is a radioactive, coloured, bioluminescent, chemiluminescent, fluorescent or phosphorescent label.

7. The conjugate of claim 6, wherein the detectable label is a fluorescent dye or tautomers thereof.

8. The conjugate of claim 7, wherein the adsorption maximum of the fluorescent dye is 600 to 900 nm.

9. The conjugate of claim 7, wherein the fluorescent dye is selected from Cy 5, Cy 5.5, Cy 7, C 3, Cy 3.5, flourescein (FITC), heptamethylene thiocyanine, ROX, TAMRA, CAL Red, Red 640, FAM, TET, HEX, Oregon Green, TRITC, APC, DY-751, ATTO 740, ATTO 725 and ATTO 700.

10. The conjugate of claim 1, wherein the detectable label is Fe-oxide, Gd-chelate.

11. The conjugate of claim 1, wherein the detectable label is compound selected from the group of ^{99m}Tc-, ⁶⁸Ga-chelators or from the group of the radioactive compounds such as ¹⁸F, ¹²⁵I.

12. The conjugate of claim 1 for use in in vivo imaging of aminopeptidase N and/or of APN-associated diseases.

13. The conjugate of claim 1 for use in the treatment of APN-associated diseases.

14. A diagnostic composition comprising the conjugate of claim 1 and optionally a diagnostic acceptable carrier.

15. A therapeutic composition comprising the conjugate of claim 1 and optionally a therapeutic acceptable carrier.

16. The composition of claim 14 for use in the diagnosis of APN-associated diseases.

17. The composition of claim 15 for use in the treatment of APN-associated diseases.

18. The conjugate of claim 13, wherein said APN-associated disease is selected from angiogenesis in cancer and inflammatory diseases, rheumatoid arthritis, leukemia and diabetic nephropathy.

19. A diagnostic or therapeutic kit comprising a conjugate of claim 1 and optionally an instruction manual.
